(19) United States
(12) Patent Application Publication
(10) Pub. No.: US 2026/0250615 A1
(43) Pub. Date: Aug. 27, 2026

(54) GAS-LIQUID SEPARATING DEVICE AND BIONIC SYSTEM

(71) Applicant: DARWIN PRECISIONS CORPORATION, Hsinchu County (TW)

(72) Inventors: CHIEH LUO, Hsinchu County (TW); Chia-Yi YEH, Hsinchu County (TW)

(21) Appl. No.: 19/547,724

(22) Filed: Feb. 24, 2026

(30) Foreign Application Priority Data
Feb. 27, 2025 (TW) 114107531

(51) Int. Cl. C12M 3/00; B01D 53/00; C12M 1/26

(52) U.S. Cl. CPC C12M 21/08; B01D 53/002; C12M 33/04; C12M 33/08

(57) ABSTRACT

A gas-liquid separating device is used in a bionic system. The gas-liquid separating device includes a container, a sprayer and a tubular cover. The container has a storage space and a gas vent, and the storage space is connected to the gas vent. The sprayer is connected to the container and is adapted for providing an aerosol to the storage space. The tubular cover is arranged in the storage space and covers the gas vent. A wall surface of the tubular cover is provided with a plurality of through holes. A bionic system using the gas-liquid separating device is also provided.

### FIELD OF THE INVENTION

The present invention relates to a gas-liquid separating device, and more particularly to a gas-liquid separating device for use in a bionic system and a bionic system using the same.

### BACKGROUND OF THE INVENTION

In the field of medical drug development, particularly in the study of physiological factors or drug research, animal testing is typically conducted prior to human trials to ensure actual effectiveness. However, due to physiological differences between animals and humans, as well as potential variations in the lifestyles of test subjects, such experiments often introduce errors and incur significant costs. As a result, the relevant industry has been attempting to develop organs-on-chips (OoC) technologies that mimic human organ tissues, as well as bionic systems that place such organs-on-chips in artificial environments to simulate human physiological conditions.

There are various types of organs-on-chips and related bionic systems, and potential simulating targets may include, for example, skin, gastrointestinal tract, liver, lungs, and so on. Among these, bionic systems simulating the lungs are particularly challenging, as they must simulate not only blood supply but also gas supply. Therefore, further development in this area is required.

### SUMMARY OF THE INVENTION

The present invention provides a gas-liquid separating device, which is applied in a bionic system to prevent liquid from condensing in the pipeline supplying gas to the organ-on-a-chip, thereby improving the reliability of the bionic system.

In order to achieve the aforementioned objects, a gas-liquid separating device for use in a bionic system is provided according to one embodiment of the present invention. The gas-liquid separating device includes a container, a gas vent tube, a sprayer, and a tubular cover. The container includes a storage space and a mounting hole. The gas vent tube is disposed in the mounting hole and includes a gas vent in communication with the storage space. The sprayer is connected to the container and provides an aerosol to the storage space. The tubular cover is disposed in the storage space and covers the gas vent. A wall surface of the tubular cover is provided with a plurality of through holes.

In an embodiment, a surface of the tubular cover includes a hydrophobic layer.

In an embodiment, the hydrophobic layer includes a biocompatible material.

In an embodiment, the container includes an upper cover, and the tubular cover tapers from an end adjacent to the upper cover toward the other end.

In an embodiment, an end of the tubular cover away from the upper cover is a closed end.

In an embodiment, the through holes have a diameter between 1 mm and 5 mm.

In an embodiment, a spacing between the through holes is between 0.4 mm and 0.6 mm.

In an embodiment, the container further includes a gas inlet in communication with the storage space.

According to an embodiment of the present invention, a gas-liquid separating device for use in a bionic system is provided. The gas-liquid separating device includes a container, a sprayer, and a tubular cover. The container includes a storage space and a gas vent. The storage space is in communication with the gas vent. The sprayer is connected to the container and adapted to provide an aerosol to the storage space. The tubular cover is disposed within the storage space and covers the gas vent. A wall surface of the tubular cover is provided with a plurality of through holes.

According to an embodiment of the present invention, a bionic system including an organ-on-a-chip, a gas supply source, and the aforementioned gas-liquid separating device is provided. The gas-liquid separating device is in communication with the organ-on-a-chip, and the gas supply source is in communication with the gas-liquid separating device.

As described above, since the gas-liquid separating device of the present invention provides a tubular cover with through holes disposed at the gas vent of the container, when the aerosols with different sizes are generated by the sprayer, the larger aerosols, which are more prone to condensation, first condense on the tubular cover and are collected inside the container, and only the smaller aerosols may pass toward the organ-on-a-chip. This effectively prevents aerosol condensation into water droplets within the pipeline leading to the organ-on-chip, which would otherwise obstruct gas supply to the organ-on-chip, thereby enhancing the reliability of the bionic system using the gas-liquid separating device of the present invention.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic diagram of a bionic system according to an embodiment of the present invention;

FIG. 2 is a schematic diagram of the gas-liquid separating device in FIG. 1;

FIG. 3 is a schematic cross-sectional view of the tubular cover in FIG. 2;

FIG. 4 is a schematic diagram of the through holes of the tubular cover in FIG. 2; and

FIG. 5 is a schematic diagram of a gas-liquid separating device according to another embodiment of the present invention.

### DETAILED DESCRIPTION OF PREFERRED EMBODIMENTS

In the following description of embodiments according to the present invention, terms indicating orientation or positional relationships, such as “upper,” “lower,” etc., are described based on the orientation or positional relationships shown in the corresponding drawings. These terms are used solely for the convenience of describing the invention and are not intended to limit the invention or to imply that the referenced elements must have a particular orientation or be constructed in a particular orientation. Furthermore, the terms “first,” “second,” and the like, as used in this specification or the claims, are employed merely to designate names of elements or to distinguish between different embodiments or ranges and are not intended to limit the number of elements. Unless limited otherwise, the terms “connected” or “coupled” as used in this specification or the claims may refer to either a direct connection between two elements without the presence of other elements or an indirect connection through the presence of other elements.

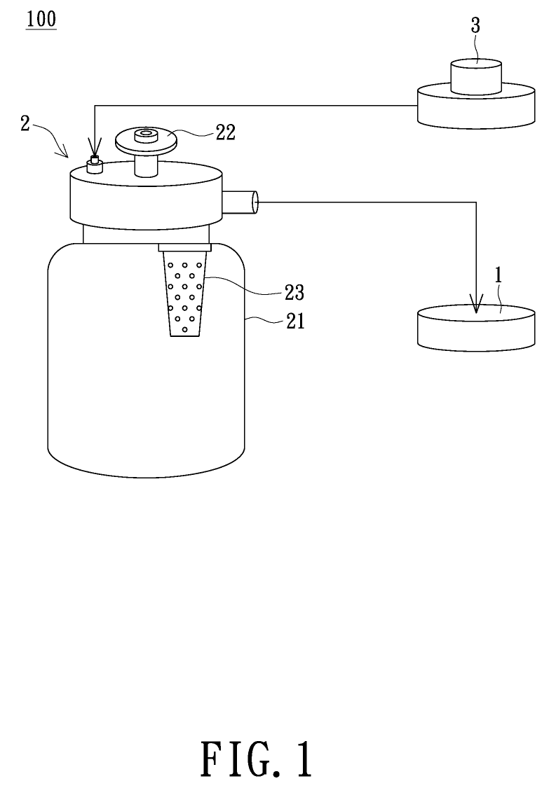

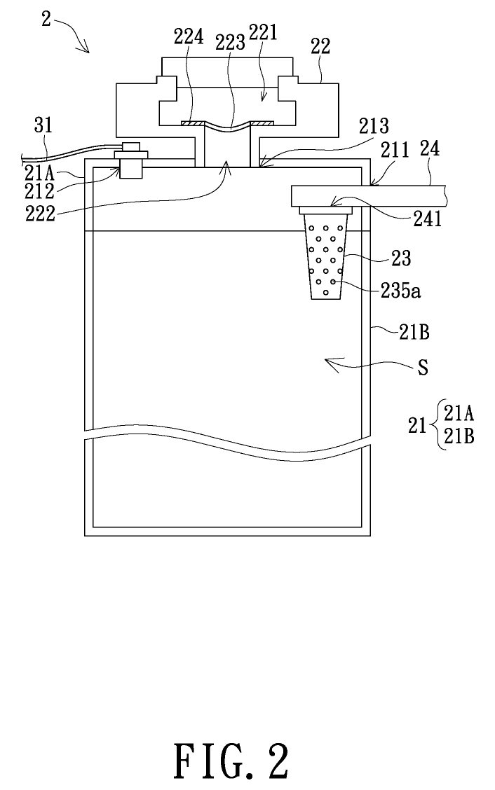

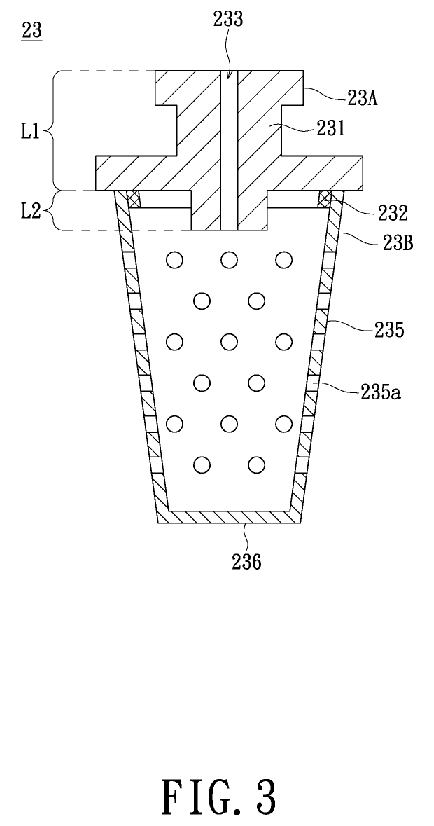

FIG. 1 is a schematic diagram of a bionic system according to an embodiment of the invention. FIG. 2 is a schematic diagram of a gas-liquid separating device in FIG. 1. FIG. 3 is a schematic cross-sectional view of a tubular cover in FIG. 2.

As shown in FIG. 1 to FIG. 3, according to an embodiment of the present invention, a bionic system 100, including an organ-on-a-chip 1, a gas-liquid separating device 2, and a gas supply source 3, is provided. The gas-liquid separating device 2 is in communication with the organ-on-a-chip 1 and includes a container 21, a gas vent tube 24, a sprayer 22, and a tubular cover 23. The container 21 includes a storage space S and a mounting hole (e.g., a first mounting hole 211). The gas vent tube 24 is disposed in the mounting hole (first mounting hole 211) and includes a gas vent 241 in communication with the storage space S. The sprayer 22 is connected to the container 21 and adapted to provide an aerosol to the storage space S. The tubular cover 23 is arranged within the storage space S and covers the gas vent 241, wherein a wall surface 235 of the tubular cover 23 is provided with a plurality of through-holes 235a. The gas supply source 3 is in communication with the gas-liquid separating device 2.

The type of the bionic system 100, for example, is determined according to the type of the organ-on-a-chip 1 and the required culture environment. In the embodiment, the organ-on-a-chip 1, for example, is a device for culturing a human lung tissue, and the bionic system 100, for example, is a system for simulating the human lung environment. In other embodiments of the present invention, the organ-on-a-chip 1 and the bionic system 100 may also be devices and systems for simulating human tissues, such as the respiratory tract, that can be exposed to environmental gases.

In an embodiment of the present invention, in addition to the aforementioned gas supply source system that simulates human lung respiration through the gas supply source 3 and the gas-liquid separating device 2 having the sprayer 22, the bionic system 100 may further include a liquid supply system not illustrated, which is used to provide liquid to the organ-on-a-chip 1 in order to simulate the pulmonary blood circulation system.

Furthermore, in the embodiment, the gas supply source 3 is configured to drive the aerosols suspended in the storage space S into the gas vent 241. The gas supply source 3 is in communication with the gas-liquid separating device 2, for example, through a gas delivery pipe 31. The type of the gas supply source 3 is not particularly limited and may be, for example, a gas cartridge or a compressor. The gas supply source 3 may provide gas, for example, at a constant pressure, but the invention is not limited thereto. The gas provided by the gas supply source 3 may be air, an inert gas, or a mixture of gases with specific ingredients. The gas pressure can be adjusted as required, for example, to simulate pulmonary respiratory pressure.

As shown in FIG. 2, in the embodiment, the container 21, for example, includes an upper cover 21A and a bottle body 21B.

The bottle body 21B forms the storage space S of the container 21 and is adapted for collecting the aerosols that do not enter the gas vent 241 or the condensates of the aerosols. A shape of the bottle body 21B is not particularly limited and may, for example, be a cylindrical container 21. A material of the bottle body 21B is not particularly limited and may, for example, be a material that allows a user to directly observe the storage space S for facilitating removing the condensates, such as glass.

The upper cover 21A is adapted to cover the bottle body 21B and connected to the gas delivery pipe 31, the sprayer 22, and the gas vent tube 24 which is in communication with the organ-on-a-chip 1. As shown in FIG. 2, in the present embodiment, the gas vent tube 24, for example, is inserted into the storage space S laterally from a side of the upper cover 21A through the first mounting hole 211 formed in a sidewall of the upper cover 21A. The invention is not limited thereto.

In an embodiment of the present invention, the gas and aerosols in the storage space S pass through the gas vent 241 from the storage space S and then move to the organ-on-a-chip 1. The configuration and the position of the gas vent 241 may be adjusted based on the requirement. In the embodiment of the present invention, the gas vent 241 may be disposed on the gas vent tube 24 or the upper cover 21A (described in detail as below). After passing through the gas vent 241 of the gas vent tube 24 or the gas vent 241 on the upper cover 21A, the gas and aerosols in the storage space S enter the gas vent tube 24 and then move to the organ-on-a-chip 1. The invention is not limited thereto. A material of the upper cover 21A is not particularly limited. For example, the upper cover 21A may be made of a material resistant to biological contamination.

In the present embodiment, the upper cover 21A, for example, includes a gas inlet 212 in communication with the gas supply source 3, a first mounting hole 211 for installing the gas vent tube 24 which passes therethrough, and a second mounting hole 213 for installing the sprayer 22. The first mounting hole 211 is, for example, disposed in a sidewall of the upper cover 21A. The second mounting hole 213 and the gas inlet 212 are disposed on a top of the upper cover 21A (i.e., the top of the storage space S).

An end of the gas vent tube 24, which is inserted through the first mounting hole 211, may be a closed end. The gas vent 241 may be disposed in a sidewall of the gas vent tube 24 at the end extending through the first mounting hole 211. An extending direction of the gas vent 241 may be different from an extending direction of the gas vent tube 24, which is disposed in and inserted through the first mounting hole 211. However, the extending direction of the gas vent 241 is the same as an extending direction of the second mounting hole 213 and an extending direction of the gas inlet 212, such as a direction toward a bottom of the bottle body 21B (i.e., a bottom of the storage space S). The invention is not limited thereto. As such, it helps to prevent the gas or the aerosols entering the storage space S from being directly sprayed toward the gas vent 241.

In the present embodiment, the sprayer 22 is, for example, disposed between the gas vent 241 and the gas inlet 212. By placing the second mounting hole 213, where the aerosols are provided to the storage space S, and the gas inlet 212 at different locations, the present embodiment prevents the gas flow from the gas inlet 212 from blowing directly toward the second mounting hole 213 and the gas vent 241. However, the connection relationship between the gas inlet 212 and the sprayer 22 in the invention is not limited thereto. In an embodiment not illustrated, the gas inlet 212 may be disposed in the wall where the second mounting hole 213 is located at or in the flow path of the sprayer 22, such that the gas supplied by the gas supply source 3 can enter the storage space S through the second mounting hole 213.

As shown in FIG. 2, the sprayer 22 in the embodiment, for example, includes a storage tank 221 in the top, a channel 222 located under the storage tank 221 and in communication with the storage space S, a vibrating sheet 223 disposed between the channel 222 and the storage tank 221, and a piezoelectric component 224 disposed in the bottom of the storage tank 221 and connected to the vibrating sheet 223 to be adapted for triggering the vibrating sheet 223, though the type of the sprayer 22 of the present invention may be selected based on the requirements.

The storage tank 221 is adapted to store liquid for making the aerosol. The type and the composition of the liquid can be determined based on the environment to be simulated by the bionic system 100 or the content of the experiment, and may include, for example, water, suspended particulates, irritating chemicals, or pharmaceutical ingredients. After the liquid passes through the sprayer, the aerosols containing the aforementioned ingredients are then formed.

The piezoelectric component 224 is connected to the vibrating sheet 223 and a power supply (not illustrated) and adapted to drive the vibrating sheet to vibrate. The type of the piezoelectric component 224 may be selected based on the requirements.

The vibrating sheet 223, for example, is a thin sheet having a plurality of small holes (not shown). A diameter of the small hole, for example, is determined based on the physical properties of the liquid in the storage tank 221, such as viscosity or surface tension, allowing the liquid in the storage tank 221 to pass through the small holes of the vibrating sheet 223 into the channel 222 and form aerosols only when the vibrating sheet 223 is vibrating.

Accordingly, in the embodiment, the sprayer 22 does not require an additional gas supply during the aerosol manufacturing process and is less likely to cause a change in the gas pressure within the storage space S.

As shown in FIG. 2 and FIG. 3, in the embodiment, the tubular cover 23, for example, is a tubular body extending from one end adjacent to the upper cover 21A to the other end in an opposite direction. Though the extending length of the tubular cover 23 is not particularly limited, for example, it may be lower than the opening of the channel 222 of the sprayer 22 and does not contact the bottom of the storage space S. The inner diameter and outer diameter of the tubular cover 23 may be determined according to the manufacturing requirements. However, the inner diameter, for example, corresponds to and is greater than a size of the gas vent 241. A thickness of the wall surface 235 of the tubular cover 23 is, for example, between 0.8 mm and 2 mm. Specifically, the thickness may be 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, or 2 mm, but is not limited thereto.

As shown in FIG. 3, in the embodiment, the tubular cover 23, for example, includes a first part 23A and a second part 23B.

The first part 23A is, for example, a tubular shape. The first part 23A includes a neck 231 and an assembly block 232. The neck 231 includes, for example, a first section L1 and a second section L2 along an extending direction. For example, an outer diameter of the first section L1 is greater than an outer diameter of the second section L2. The neck 231 is adapted to be fitted on the gas vent 241. The first part 23A includes a vent hole 233 penetrating through the neck 231 (the first section L1 and the second section L2).

In the embodiment, the first part 23A is provided with the assembly block 232 disposed in one end of the first section L1 of the neck 231, which is adjacent to the second section L2 of the neck 231. The assembly block 232, for example, is ring-shaped and disposed surrounding the second section L2 of the neck 231. The radial inner wall surface of the assembly block 232, for example, is separated from the second section L2 of the neck 231. The radial outer wall surface of the assembly block 232 is adapted to be fitted on the inner wall surface of the second part 23B.

The second part 23B, for example, is fitted with the assembly block 232. The second part 23B, for example, is a barrel-shaped tubular body that gradually tapers from an end adjacent to the upper cover 21A (the top of the container 21) toward the opposite end. The wall surface 235 of the second part 23B (e.g., the sidewall of the second part 23B) is provided with a through hole 235a. An extending direction of the through hole 235a is, for example, but not limited to, different from the extending direction of the tubular body of the second part 23B (e.g., perpendicular).

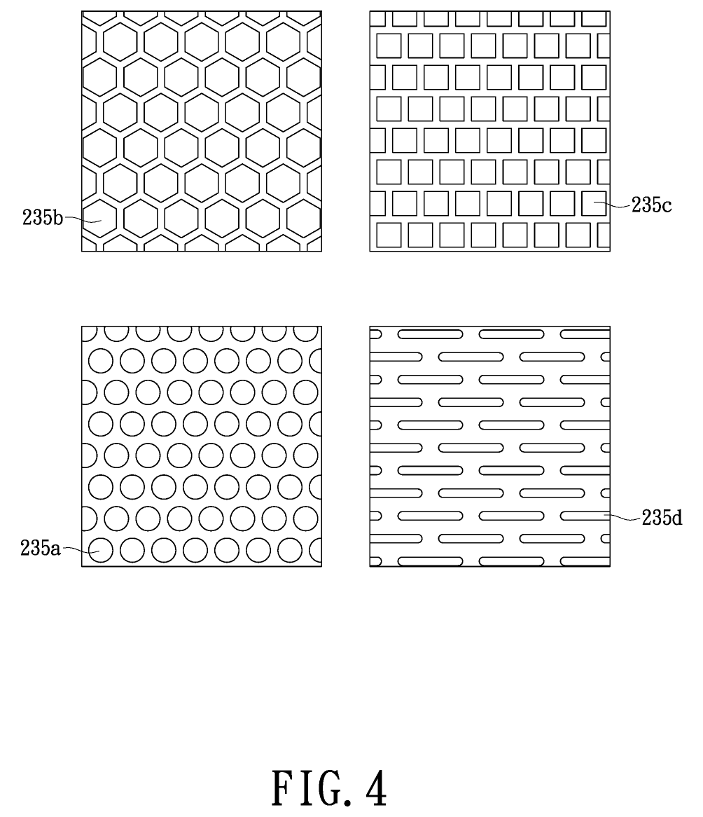

FIG. 4 is a schematic diagram of the through holes of the tubular cover in FIG. 2. As shown in FIG. 3 and FIG. 4, a shape of the through hole 235a is not particularly limited and may be one of a circular shape (as the through hole 235a in FIG. 4), a hexagonal shape (as the through hole 235b in FIG. 4), a square shape (as the through hole 235c in FIG. 4), and a strip shape (as the through hole 235d in FIG. 4), or a combination thereof.

In an embodiment of the present invention, the through holes 235a of the second parts 23B located on different sides, for example, are oriented toward each other, such that the through holes 235a on the different sides extend along the same direction and are aligned in the same straight line. In another embodiment, the through holes 235a on the different sides extend in the same direction but are aligned in the different straight lines. A diameter of the through hole 235a is, for example, less than a diameter of the vent hole 233 and between 1 mm and 5 mm, for example, but not limited to 1 mm, 1.5 mm, 2 mm, 2.5 mm, 3 mm, 3.5 mm, 4 mm, 4.5 mm, or 5 mm. A spacing between the two adjacent through holes 235a may be, for example, between 0.4 mm and 0.6 mm, and may be, for example, but not limited to, 0.4 mm, 0.45 mm, 0.5 mm, 0.55 mm, or 0.6 mm.

In the present embodiment, the second part 23B includes, for example, an end surface 236 at an end of the second part 23B away from the upper cover 21A. The end surface 236, for example, does not include the through hole 235a and is a closed end. However, the invention is not limited thereto. In some embodiments, the tubular cover 23 may be a cone or a bullet shape without the end surface 236.

In the present embodiment, the tubular cover 23 may be made of a material including one of stainless steel, iron, aluminum, plastic, or a combination thereof. For example, the tubular cover 23a may include a hydrophobic layer covering on the surface of the tubular cover 23 (e.g., the inner and outer surface of the second part 23B and the wall surface of the through hole 235a). The hydrophobic layer has a contact angle, for example, greater than 95 degrees. The hydrophobic layer, for example, includes a layer made of a biocompatible material, such as parylene, but is not limited thereto.

The tubular cover 23 is in communication with the organ-on-a-chip 1 and the container 21 through the vent hole 233 of the first part 23A and the through holes 235a of the second part 23B. As shown in FIG. 3, in the embodiment, the extending direction of the vent hole 233 is different from the extending direction of the through holes 235a (e.g., perpendicular). However, the invention is not limited thereto.

Accordingly, by using the through holes 235a, the tapered tubular body, and the hydrophobic layer, the tubular cover 23 enables rapid and directional removal of the liquid condensed on the wall surface 235 of the tubular cover 23, thereby preventing the through holes 235a from being blocked. Accordingly, the condensed aerosols removed by the tubular cover 23 may be collected within the storage space S.

It should be understood that, in one embodiment of the present invention, the extending direction of the gas inlet 212 and the extending direction of the sprayer 22 are, for example, not intersecting with the extending direction of the gas vent 241. The aerosols are indirectly driven to enter the tubular cover 23 by the gas flow generated by the gas supply source 3, thereby preventing the larger aerosols, which are likely to condense in the gas vent tube 24, from entering the gas vent tube 24. In addition, compared with the embodiment shown in FIG. 2, in an embodiment not illustrated, the gas inlet 212 and the sprayer 22 may be disposed in a sidewall of the container 21, and the first mounting hole 211 may be disposed at the top of the upper cover 21A, such that the extending directions of the gas inlet 212, the sprayer 22, and the first mounting hole 211 are partially intersecting with each other. It is noted that the foregoing is merely exemplary, and the present invention imposes no particular limitation on details, such as whether the gas inlet 212 and the sprayer 22 are oriented toward the gas vent 241.

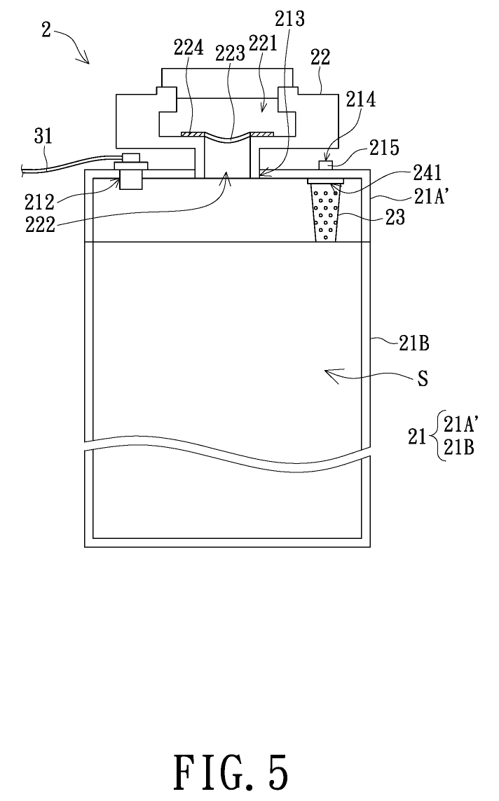

FIG. 5 is a schematic diagram of a gas-liquid separating device according to another embodiment of the present invention. As shown in FIG. 5, in the embodiment, the tubular cover 23 and the sprayer 22 are designed in the same way as the aforementioned embodiments except the shape of the upper cover 21A’ of the container 21 and the connection of the gas vent tube 24.

Specifically, as shown in FIG. 5, the upper cover 21A’ is provided with a gas vent 214, which is disposed on the top wall of the upper cover 21A’ and penetrates through the upper cover 21A’. The tubular cover 23 covers the gas vent 214. The extending direction of the gas vent 214 is, for example, towards the bottom of the container 21. The upper cover 21A’, for example, is provided with an installation tube 215 extended from the surface of the outer wall and in communication with the gas vent 214. The installation tube 215 is adapted to connect to the gas vent tube 24 (not shown in FIG. 5). The extending direction of the installation tube 215 is not particularly limited and may be configured based on the requirements. The second mounting hole 213, the gas inlet 212, and the gas vent 241 are disposed, for example, on the same side of the upper cover 21A’. Specifically, they may be disposed at the top of the upper cover 21A’ (i.e., the top of the storage space S).

Similar to the embodiment in FIG. 5, in one embodiment where the gas vent tube 24 does not enter the container 21, the gas vent 214 may be disposed in the sidewall of the upper cover 21A’ by changing the shape of the first part 23A of the tubular cover 23 (e.g., L-shaped).

As described above, in the gas-liquid separating device of the present invention, a tubular cover having through holes is disposed at the gas vent of the container. Among the various sizes of aerosols produced by the sprayer, the larger aerosols, which are more likely to condense, can first condense on the tubular cover and then be collected within the container, and only the smaller aerosols are allowed to pass toward the organ-on-a-chip. As a result, condensation of the aerosols into water droplets within the pipelines to the organ-on-a-chip is prevented, whereby obstruction of the gas supply to the organ-on-a-chip is avoided, and the reliability of the bionic system using the gas-liquid separating device of the present invention is improved.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A gas-liquid separating device for use in a bionic system, the gas-liquid separating device comprising:
a container, comprising a storage space and a mounting hole;
a gas vent tube, disposed in the mounting hole and comprising a gas vent, wherein the gas vent is in communication with the storage space;
a sprayer, connected to the container and adapted to provide an aerosol to the storage space; and
a tubular cover, disposed within the storage space and covering the gas vent, wherein a wall surface of the tubular cover comprises a plurality of through holes.

2. The gas-liquid separating device according to claim 1, wherein a surface of the tubular cover comprises a hydrophobic layer.

3. The gas-liquid separating device according to claim 2, wherein the hydrophobic layer comprises a biocompatible material.

4. The gas-liquid separating device according to claim 1, wherein the container comprises an upper cover, and the tubular cover tapers from a first end toward a second end, the first end is relatively adjacent to the upper cover, and the second end is relatively far from the upper cover.

5. The gas-liquid separating device according to claim 4, wherein an end of the tubular cover away from the upper cover is a closed end.

6. The gas-liquid separating device according to claim 1, wherein the through holes have a diameter between 1 mm and 5 mm.

7. The gas-liquid separating device according to claim 1, wherein a spacing between the through holes is between 0.4 mm and 0.6 mm.

8. The gas-liquid separating device according to claim 1, wherein the container further comprises a gas inlet in communication with the storage space.

9. A gas-liquid separating device for use in a bionic system, the gas-liquid separating device comprising:
a container, comprising a storage space and a gas vent, wherein the storage space is in communication with the gas vent;
a sprayer, connected to the container and configured to provide an aerosol to the storage space; and
a tubular cover, disposed within the storage space and covering the gas vent, wherein a wall surface of the tubular cover is provided with a plurality of through holes.

10. The gas-liquid separating device according to claim 9, wherein a surface of the tubular cover comprises a hydrophobic layer.

11. The gas-liquid separating device according to claim 10, wherein the hydrophobic layer comprises a biocompatible material.

12. The gas-liquid separating device according to claim 9, wherein the container comprises an upper cover, and the tubular cover tapers from an end adjacent to the upper cover toward the other end.

13. The gas-liquid separating device according to claim 12, wherein an end of the tubular cover away from the upper cover is a closed end.

14. The gas-liquid separating device according to claim 9, wherein the container comprises an upper cover and a bottle body, and the gas vent is disposed on the upper cover.

15. The gas-liquid separating device according to claim 9, wherein the through holes have a diameter between 1 mm and 5 mm.

16. The gas-liquid separating device according to claim 9, wherein a spacing between the through holes is between 0.4 mm and 0.6 mm.

17. The gas-liquid separating device according to claim 9, wherein the container further comprises a gas inlet in communication with the storage space.

18. A bionic system, comprising:
an organ-on-a-chip;
a gas-liquid separating device, in communication with the organ-on-a-chip, the gas-liquid separating device comprising:
a container, comprising a storage space and a mounting hole;
a gas vent tube, disposed in the mounting hole and comprising a gas vent, wherein the gas vent is in communication with the storage space;
a sprayer, connected to the container and adapted to provide an aerosol to the storage space; and
a tubular cover, disposed within the storage space and covering the gas vent, wherein a wall surface of the tubular cover is provided with a plurality of through holes; and
a gas supply, in communication with the gas-liquid separating device.
